(19) United States
(12) Patent Application Publication
(10) Pub. No.: US 2008/0138277 A1
(43) Pub. Date: Jun. 12, 2008

(54) Delivery systems and methods for diagnosing and treating cardiovascular diseases

(75) Inventors: Stephen E. Epstein, Rockville, MD (US); Mary Susan Burnett, Fairfax, VA (US); Martin A. Mullins, Bethesda, MD (US)

Correspondence Address: ROPES & GRAY LLP, PATENT DOCKETING 39/41, ONE INTERNATIONAL PLACE, BOSTON, MA, 02110-2624, US

(73) Assignees: MEDSTAR HEALTH INC, Columbia, MD (US); Georgetown University, Washington, DC (US)

(21) Appl. No.: 11/386,513

(22) Filed: Mar. 23, 2006

Related U.S. Application Data
(60) Provisional application No. 60/664,330, filed on Mar. 23, 2005.

(51) Int. Cl. A61K 51/00; A61K 47/42; A61K 47/06; A61K 31/70; C12N 15/00; A61K 39/395; A61K 38/00; A61K 49/00

(52) U.S. Cl. 424/1.61; 514/773; 514/772; 514/2; 424/130.1; 514/44; 424/9.1; 435/320.1

(57) ABSTRACT

The invention relates to the treatment and prevention of atherosclerosis and cardiovascular diseases associated with atherosclerosis. The invention further relates to methods of diagnosing atherosclerosis and cardiovascular diseases associated with atherosclerosis. In certain embodiments, the invention provides biological systems and methods for delivering a therapeutic agent or an imaging agent to atherosclerotic lesions such as vulnerable plaques.

### RELATED APPLICATION

This application claims the benefit of U.S. Provisional Application No. 60/664,300 filed Mar. 22, 2005. The entire teachings and specification of each of the referenced applications are incorporated herein by reference.

### BACKGROUND OF THE INVENTION

Atherosclerosis is an inflammatory disease, eventually leading to an accumulation of lipids within the artery wall. Injury to the vascular endothelium, by hypercholesterolemia, hypertension, elevated levels of homocysteine, infection, and/or other conditions, appears to be the initiating event of atherosclerosis. This causes arterial endothelial cells to express adhesion molecules that bind various classes of leukocytes, including monocytes and T lymphocytes. Once adherent to the endothelium, chemokines (such as MCP-1) expressed within the injured arterial wall stimulate the leukocytes to migrate into the intima and thereby establish an inflammatory process ultimately leading to the development of the typical atherosclerotic plaque. This includes the transdifferentiation of monocytes into macrophages. These cells express scavenger receptors that lead to the intracellular accumulation of lipids and thereby cause the macrophages to become foam cells—the beginning of the lipid accumulation component of the atherogenic plaque.

Inflammation also contributes to the precipitation of the characteristic acute thrombotic complications of atherosclerosis. A thick fibrous cap containing collagen normally separates the lipid constituents of the plaque from the circulating blood. However, if the inflammatory cells resident within the plaque are activated, they can produce proteolytic enzymes that degrade the cap's collagen, causing it to become susceptible to erosion and rupture. Exposure of the prothrombotic constituents of the plaque to the circulating blood initiates thrombotic processes, which are further facilitated by inflammatory molecules inducing macrophages to express tissue factor, a potent trigger of thrombosis.

Increasing knowledge of the pathogenesis of atherosclerosis suggests that prevention of cardiovascular disease will involve not only the correction of the above risk factors, but also the direct pharmacological control of atherogenic processes occurring in the arterial wall (Ross, R., Nature, 362:801-809 (1993)). Accordingly, a need exists to develop novel therapies to treat and prevent vascular diseases and atherosclerosis by regulating the atherogenic process and to develop novel methods for diagnosis applications.

### SUMMARY OF THE INVENTION

The present invention relates to biological systems for delivering an agent, such as a therapeutic or diagnostic agent, to atherosclerotic lesions in blood vessels in an individual; the atherosclerotic lesions are, for example, stable atherosclerotic plaques or vulnerable atherosclerotic plaques. It further relates to uses of the biological systems, for example, in treating individuals in need of therapy, in methods of delivering an imaging agent(s) to atherosclerotic lesions in an individual and in diagnosis or aiding in the diagnosis of atherosclerosis and/or the presence of atherosclerotic plaques in an individual.

In certain embodiments, the invention provides a biological system for delivering a therapeutic agent to atherosclerotic lesions (e.g., stable atherosclerotic plaques, vulnerable atherosclerotic plaques) in blood vessels in an individual. Such biological system comprises: (a) a therapeutic agent and (b) a carrier that comprises at least one targeting moiety that interacts with a molecule present in atherosclerotic lesions, referred to herein as a “target molecule,” wherein the biological system delivers the therapeutic agent to atherosclerotic lesions in blood vessels. In further embodiments, the biological system comprises two or more targeting moieties that interact with different target molecules present in atherosclerotic lesions. The multiple targeting moieties may be on the same carrier or on different carriers. Certain examples of the target molecules present in atherosclerotic lesions include, but are not limited to, ligands such as CXCL1 (growth-regulated oncogene-alpha); CXCL5 (ENA-78); CXCL8 (IL-8); CXCL9 (Mig); CXCL10 (IP-10); CXCL11 (I-TAC); CXCL12 (SDF-1); CCL2 (MCP-1); CCL3 (MIP-1alpha); CCL5 (RANTES); CCL7 (MCP-3); CCL17 (TARC); CCL22 (MDC); fractalkine (FKN); HSP60, (S)-2-amino-4-phosphonobutanoic acid and (S)-3,4-dicarboxyphenylglycine; IL-6; IL-8; IFN-γ; or TNF-α. Target molecules can also be adhesion molecule ligands such as VCAM-1 and ICAM-1. In this case, the targeting moiety of the biological system may be a receptor of any one of these ligands. Alternatively, the targeting moiety can be an antibody that binds to the ligand. Other examples of the target molecules present in atherosclerotic lesions include, but are not limited to, receptors of the ligands, such as a receptor of CXCL1 (growth-regulated oncogene-alpha); CXCL5 (ENA-78); CXCL8 (IL-8); CXCL9 (Mig); CXCL10 (IP-10); CXCL11 (I-TAC); CXCL12 (SDF-1); CCL2 (MCP-1); CCL3 (MIP-1alpha); CCL5 (RANTES); CCL7 (MCP-3); CCL17 (TARC); CCL22 (MDC); fractalkine (FKN); HSP60, (S)-2-amino-4-phosphonobutanoic acid and (S)-3,4-dicarboxyphenylglycine); IL-6, IL-8, IFN-γ, or TNF-α. To illustrate, the receptors include, but are not limited to, CXCR3, CXCR4, CCR4, CX3CR1, toll-like receptors, and metabotropic glutamate receptors (mGluRs). In this case, the targeting moiety of the biological system may be a corresponding ligand that binds to the receptor. Alternatively, the targeting moiety can be an antibody that binds to the receptor. Further examples of the target molecules present in atherosclerotic lesions include constituents of the atherosclerotic lesions, such as lipids and extracellular matrix. In this case, the targeting moiety of the biological system can be an antibody that binds to an extracellular matrix molecule (e.g., proteoglycan or collagen) or a lipid-binding agent (e.g., oil red). Osteopontin can also be a target molecule.

In one specific embodiment, this biological system further comprises a second carrier that comprises a therapeutic agent and a binding partner that interacts with a component/constituent of the carrier (referred to as the first carrier) that comprises the targeting moiety. Through the interaction between the binding partner and the targeting moiety, the second carrier is attracted to the lesion sites by the first carrier, thereby leading to increased delivery of the therapeutic agent.

The therapeutic agents in the biological system may be a nucleic acid, a polypeptide, an antibody, a small molecule compound, and/or a peptidomimetic. In certain specific embodiments, the therapeutic agents are polypeptides (which include peptides and proteins) or the nucleic acids encoding a polypeptide(s) to be delivered. In specific embodiments, therapeutic agents are nucleic acids that either consist of naked DNA or are present in a vector, such as a viral vector inserted as a transgene into a viral vector. Examples of therapeutic agents in the biological system include, but are not limited to, (a) agents that reduce lipid levels in atherosclerotic lesions, such as an HMG-CoA reductase inhibitor, a thyromimetic, a fibrate, or an agonist of peroxisome proliferator-activated receptors (PPAR); (b) agents that reduce an oxidative process in a mammal, such as cytokine-stimulated cyclohydrolase-1 (GTPCH-1) or haptoglobin; (c) agents that modulate expression of an endothelial cell receptor, an endothelial cell adhesion molecule, an endothelial cell integrin, a smooth muscle cell receptor, a smooth muscle cell adhesion molecule or a smooth muscle cell integrin; (d) agents that modulate the proliferation of an endothelial cell or a smooth muscle cell in a mammalian blood vessel; (e) agents that modulate an inflammation associated receptor (or the ligand of such receptor, or the signaling pathway of such a receptor); or inflammation associated transcription factors, such as chemokine receptors, tissue factor (TF); RAGE receptors, toll-like receptors, angiotensin receptors, a TGF receptor, interleukin receptors, a TNF receptor, an IFNγ receptor, a metabotropic glutamate 8 (mGlu8) receptor, receptors that can activate NF-κcb; CXCL12 (SDF-1, and its receptor CXCR4); CCL17 (TARC); CCL22 (MDC); CCL3 (MIP-1alpha); CCL5 (RANTES); CCL7 (MCP-3); CXCL1 (growth-regulated oncogene-alpha); CXCL5 (ENA-78); CXCL8 (IL-8); fractalkine (FKN; and its receptor, CX3CR1); HSP60, toll-like receptors, metabotropic glutamate receptors (mGluRs; and its ligands, such as (S)-2-amino-4-phosphonobutanoic acid and (S)-3,4-dicarboxyphenylglycine); c-Fos, IL-6, IL-8, MCP-1, IFN-γ, and TNF-α; (f) agents that modulate proliferation, apoptosis or necrosis of endothelial cells, vascular smooth muscle cells, lymphocytes, monocytes, or neutrophils; (g) agents that modulate production, degradation, or crosslinking of an extracellular matrix protein, such as collagen, a elastin, or a proteoglycan; (h) agents that modulate activation, secretion or lipid loading of a cell within a mammalian blood vessel; (i) agents that modulate activation or proliferation of a dendritic cell or monocyte/macrophage cell within a mammalian blood vessel; (j) agents that modulate activation or adhesion of a platelet at a mammalian blood vessel wall or otherwise inhibit clot formation, such as activated protein C (APC); and (k) agents consisting of a nucleic acid that encodes a protein therapeutic agent, which has an in vivo activity that is beneficial to a mammal suffering from an atherosclerotic lesion. One specific example of the therapeutic agents is HDL, a peptide component of HDL that has similar therapeutic actions as HDL, or a nucleic acid encoding such peptide component. Their therapeutic actions include, but are not limited to, binding to cholesterol and transport to tissues in which it is degraded, and binding to a receptor involved in cholesterol transport, such as the ATP-binding cassette transporter 1 (ABCA1) or the SR-B1 receptor, which are located in macrophages and in blood vessel wall cell.

The carriers of the biological system include, but are not limited to, liposomes, micelles, cells, viral particles, viruses, nanoparticles, such as nanospheres, or microparticles, such as microspheres, chambered micro-devices, emulsions, lipid discs, polymers, gadolinium-conjugated molecules, superparamagnetic iron oxide particles, multimodal perfluorocarbon nanoparticles, and microbubbles. One or more targeting moieties may be linked to the surface of the carriers. The carriers may carry a therapeutic agent, either as the polypeptide/peptide, or a transgene that encodes one or more therapeutic polypeptide/peptide agents.

Preferably, the biological system delivers a therapeutic agent to vulnerable lesions/plaques. Such lesions/plaques are referred to as vulnerable because they are susceptible to rupture or erosion, which facilitates thrombus formation that leads to partial or total vessel occlusion. The terms “lesion” and “plaque” are used interchangeably herein. The terms “stable atherosclerotic plaque” and “stable atherosclerotic lesion” are used interchangeably herein. The terms “vulnerable atherosclerotic plaque” and “vulnerable atherosclerotic lesion” are used interchangeably herein. The term “atherosclerotic plaque” and “atherosclerotic lesion” are also used interchangeably herein and include, but are not limited to, stable atherosclerotic plaque, stable atherosclerotic lesion, vulnerable atherosclerotic plaque and vulnerable atherosclerotic lesion.

In certain further embodiments, the invention provides a biological system for delivering an imaging agent (one or more imaging agent) to atherosclerotic lesions in blood vessels in an individual. Such biological system comprises: (a) an imaging agent and (b) a carrier that comprises at least one targeting moiety that interacts with a target molecule present in atherosclerotic lesions, wherein the biological system delivers the imaging agent to atherosclerotic lesions in blood vessels. In further embodiments, the biological system comprises two or more targeting moieties that interact with different target molecules present in atherosclerotic lesions. The multiple targeting moieties may be on the same carrier or on different carriers. Certain examples of the target molecules present in atherosclerotic lesions include, but are not limited to, ligands such as CXCL1 (growth-regulated oncogene-alpha)-CXCL5 (ENA-78); CXCL8 (IL-8); CXCL9 (Mig); CXCL10 (IP-10); CXCL11 (1-TAC); CXCL12 (SDF-1); CCL2 (MCP-1); CCL3 (MIP-1alpha); CCL5 (RANTES); CCL7 (MCP-3); CCL17 (TARC); CCL22 (MDC); fractalkine (FKN); HSP60, (S)-2-amino-4-phosphonobutanoic acid and (S)-3,4-dicarboxyphenylglycine); IL-6, IL-8, IFN-γ, and TNF-α. Target molecules can also be such adhesion molecules as VCAM-1 and ICAM-1. In this case, the targeting moiety of the biological system may be a receptor of the ligand. Alternatively, the targeting moiety can be an antibody that binds the ligand. Other examples of the target molecules present in atherosclerotic lesions include, but are not limited to, receptors of the ligands, such as a receptor of CXCL1 (growth-regulated oncogene-alpha); CXCL5 (ENA-78); CXCL8 (IL-8); CXCL9 (Mig); CXCL10 (IP-10); CXCL11 (1-TAC); CXCL12 (SDF-1); CCL2 (MCP-1); CCL3 (MIP-1alpha); CCL5 (RANTES); CCL7 (MCP-3); CCL17 (TARC); CCL22 (MDC); fractalkine (FKN); HSP60, (S)-2-amino-4-phosphonobutanoic acid and (S)-3,4-dicarboxyphenylglycine); IL-6, IL-8, IFN-γ, or TNF-α. To illustrate, the receptors include, but are not limited to, CXCR3, CXCR4, CCR4, CX3CR1, toll-like receptors, and metabotropic glutamate receptors (mGluRs). In this case, the targeting moiety of the biological system may be a corresponding ligand that binds to the receptor. Alternatively, the targeting moiety can be an antibody that binds the receptor. Further examples of the target molecules present in atherosclerotic lesions include constituents of the atherosclerotic lesions, such as lipids and extracellular matrix. In this case, the targeting moiety of the biological system can be an antibody that binds an extracellular matrix molecule (e.g., proteoglycan, collagen) or a lipid-binding agent, such as oil red. Osteopontin can also be a target molecule.

In one specific embodiment, this biological system further comprises a second carrier that comprises an imaging agent and a binding partner that interacts with a component/constituent of the carrier (referred to as the first carrier) that comprises the targeting moiety. Through the interaction between the binding partner and the targeting moiety, the second carrier is attracted to the lesion sites by the first carrier, thereby leading to increased delivery of the imaging agent.

Examples of the imaging agents in the biological system include, but are not limited to, radioactive agents (e.g., radioiodine, technetium, yttrium, or other radiopharmaceutical); contrast agents (e.g., gadolinium, manganese, barium sulfate, iodinated or noniodinated agents, ionic agents or nonionic agents, superparamagnetic iron oxide particles, and multimodal perfluorocarbon nanoparticles); magnetic agents or paramagnetic agents; liposomes (e.g., a liposome that carries a radioactive agent, a contrast agent, or any other imaging agent as described herein); micelles, cells, viral particles, viruses, microparticles, such as microspheres, nanoparticles, such as nanospheres, chambered micro-devices, emulsions, lipid discs, polymers, gadolinium-conjugated molecules, superparamagnetic iron oxide particles, multimodal perfluorocarbon nanoparticles, and microbubbles. In certain cases, the imaging agent is a fluorescent polypeptide (e.g., luciferase) that is encoded by the expression vector or virus. Optionally, the imaging agents include those agents that have been employed for CT, fluoroscopy, SPECT imaging, optical imaging, PET, MRI or gamma imaging.

The carriers of this biological system include, but are not limited to, a liposome, a micelle, a cell, a viral particle, a virus, a nanoparticle, such as a nanosphere or a microparticle, such as a microsphere, a chambered micro-device, an emulsion, a lipid disc, a polymer, gadolinium-conjugated molecules, superparamagnetic iron oxide particles, multimodal perfluorocarbon nanoparticles, and microbubbles. One or more targeting moieties may be linked to the surface of the carriers. The carriers may carry a therapeutic agent, either as the polypeptide/peptide, or a transgene that encodes one or more therapeutic polypeptide/peptide agents.

The biological system delivers an imaging agent to vulnerable lesions/plaques. These plaques are referred to as vulnerable because they are susceptible to rupture or erosion, which facilitates thrombus formation that leads to partial or total vessel occlusion. The biological system can, alternatively, deliver an imaging agent to stable atherosclerotic plaques.

In a specific embodiment, this biological system further comprises (in addition to an imaging agent(s)) at least one therapeutic agent. The therapeutic agent and the imaging agent can be in a single carrier or separate carriers.

In certain embodiments, the invention provides a method of slowing the development of atherosclerosis in an individual, comprising administering to the individual a therapeutically effective amount of a biological system comprising (a) a therapeutic agent and (b) a carrier that comprises a targeting moiety that interacts with a target molecule present in atherosclerotic lesions, wherein the biological system delivers the therapeutic agent to atherosclerotic lesions in blood vessels. Preferably, the individual is a human. In further embodiments, the biological system administered according to the method comprises two or more targeting moieties that interact with different target molecules present in atherosclerotic lesions (e.g., stable atherosclerotic plaques, vulnerable atherosclerotic plaques). The multiple targeting moieties may be on the same carrier or on different carriers.

In certain embodiments, the invention provides a method of treating or preventing atherosclerosis in an individual, comprising administering a therapeutically effective amount of a biological system comprising (a) a therapeutic agent and (b) a carrier that comprises a targeting moiety that interacts with a target molecule present in atherosclerotic lesions, wherein the biological system delivers the therapeutic agent to atherosclerotic lesions in blood vessels. Preferably, the individual is a human. In further embodiments, the biological system administered according to the method comprises two or more targeting moieties that interact with different target molecules present in atherosclerotic lesions. The multiple targeting moieties may be on the same carrier or on different carriers. The atherosclerosis may be associated with plaque rupture, plaque erosion, acute coronary syndrome, stroke, transient ischemia attack, heart attack, angina, unstable angina, thrombosis, myocardial infarction, ischemic heart disease, peripheral artery disease, or transplantation-induced sclerosis. Optionally, this method further comprises administering at least one additional therapeutic agent, such as streptokinase, tissue plasminogen activator, plasmin, urokinase, a tissue factor protease inhibitor, a nematode-extracted anticoagulant protein, a metalloproteinase inhibitor, an anti-inflammatory agent(s), a statin, HDL (e.g., the major protein of HDL, apo A1 or a peptide component of apo A1 that has therapeutic activity similar to that of HDL; mutant apo A1, such as apoA1 Milano or other mutant form that has similar therapeutic activity).

In certain embodiments, the invention provides a method of delivering an imaging agent to atherosclerotic lesions in an individual, comprising administering to the individual an effective amount of a biological system comprising (a) an imaging agent and (b) a carrier that comprises a targeting moiety that interacts with a target molecule present in atherosclerotic lesions, wherein the biological system delivers the imaging agent to atherosclerotic lesions in blood vessels. Preferably, the individual is a human. In further embodiments, the biological system of the method comprises two or more targeting moieties that interact with different target molecules present in atherosclerotic lesions. The multiple targeting moieties may be on the same carrier or on different carriers.

In certain embodiments, the invention provides a method of identifying the severity, extent or both severity and extent of atherosclerotic lesions (stable atherosclerotic plaques, vulnerable atherosclerotic plaques or both) in an individual, comprising: 1) administering a biological system comprising (a) an imaging agent and (b) a carrier that comprises a targeting moiety that interacts with a target molecule present in atherosclerotic lesions; and 2) observing the amount, localization, shape, density, or relative distribution of the imaging agent on an atherosclerotic lesion in the individual. The targeting moiety in the biological system will be selected with reference to the method of identifying in which it will be used. For example, if the severity, extent or severity and extent of vulnerable atherosclerotic lesions/plaques is to be determined, the biological system will include a targeting moiety that interacts with a target molecule present in vulnerable atherosclerotic lesions. Similarly, if the severity, extent or severity and extent of stable atherosclerotic lesions/plaques is to be determined, the biological system will include a targeting moiety that interacts with a target molecule present in stable atherosclerotic lesions/plaques. Preferably, the individual is a human. In certain embodiments, the biological system of the method comprises two or more targeting moieties that interact with different target molecules present in atherosclerotic lesions. The multiple targeting moieties may be on the same carrier or on different carriers.

In certain specific embodiments, the invention provides a biological system for delivering an HDL therapeutic an individual. The biological system comprises: (a) an HDL therapeutic selected from HDL, apoA-1, a mutant apoA-1, an apoA-1 mimetic peptide, and a nucleic acid encoding either apoA-1 or a peptide mimetic of apoA-1; and (b) a carrier from which the HDL therapeutic of (a) is delivered to blood vessels. For example, the apoA-1 protein or an apoA-1-mimetic peptide binds to a receptor involved in cholesterol transport, such as the ATP-binding cassette transporter 1 (ABCA1) or the SR-B1 receptor. Optionally, the carrier is selected from a nanoparticle, a microparticle, a cell, and a liposome. Optionally, the nucleic acid HDL therapeutic is DNA in an expression vector.

In certain specific embodiments, the invention provides a delivery system that targets atherosclerotic plaque in blood vessels. The delivery the system comprises: (a) an HDL therapeutic selected from HDL, apoA-1, a mutant apoA-1, an apoA-1 mimetic peptide, and a nucleic acid encoding either apoA-1 or a peptide mimetic of apoA-1; and (b) a carrier that interacts/binds with a constituent of atherosclerotic plaque and delivers the HDL therapeutic of (a) to the atheroslerotic plaque. For example, the apoA-1 protein or an apoA-1-mimetic peptide binds to a receptor involved in cholesterol transport, such as the ATP-binding cassette transporter 1 (ABCA1) or the SR-B1 receptor. Optionally, the carrier is selected from a nanoparticle, a microparticle, a cell, and a liposome. Optionally, the nucleic acid HDL therapeutic is DNA in an expression vector. To illustrate, the carrier bears on its surface a binding partner of the constituent of atherosclerotic plaque, such as a constituent of the plaque itself (including molecules residing on or expressed by cells normally residing in the plaque, cells that have migrated to the plaque, extracellular matrix of the plaque, blood vessels residing in the plaque, such as vasa vasorum), or a product released/shed from the plaque. In certain cases, the atherosclerotic plaque is vulnerable to rupture.

In certain specific embodiments, the invention provides a method of slowing the development of atherosclerotic lesions in an individual, comprising administering to the individual a therapeutically effective amount of the subject biological system, whereby the development of atherosclerotic lesions is slowed in the individual.

In certain specific embodiments, the invention provides a method of reducing cholesterol levels in atherosclerotic plaque in an individual in need thereof, comprising administering to the individual a therapeutically effective amount of the subject delivery system, whereby cholesterol levels in atherosclerotic plaque are reduced in the individual.

In certain specific embodiments, the invention provides an expression vector comprising: (a) DNA that encodes a protein component of HDL selected from apoA-1, a mutant apoA-1, an apoA-1 mimetic peptide; and (b) a promoter specifically expressed in blood vessels that contain atherosclerotic plaques, wherein the DNA of (a) is operably linked to the promoter of (b). Optionally, the expression vector comprises a promoter specifically expressed in blood vessels that contain atherosclerotic plaques that are vulnerable. Merely to illustrate, one example of such a promoter would be a promoter that contains binding sites for activators usually associated with inflammatory situations (such as the promoter for MCP-1); another example would be a promoter that contains binding sites for activators usually associated with newly growing blood vessels (such as the promoter for VEGF).

### DETAILED DESCRIPTION OF THE INVENTION

The invention broadly relates to atherosclerosis and/or cardiovascular diseases associated with atherosclerosis. Some aspects of the invention provide delivery systems and methods for the treatment, prevention, and diagnosis of atherosclerosis and of cardiovascular diseases associated with atherosclerosis.

In certain embodiments, the invention provides biological delivery systems for delivering a therapeutic agent or an imaging agent to atherosclerotic lesions (stable atherosclerotic lesions or vulnerable atherosclerotic lesions) in blood vessels in an individual. In one embodiment, the biological system comprises: (a) a therapeutic agent and (b) a carrier that comprises at least one targeting moiety that interacts with a target molecule (e.g., a chemokine ligand, a chemokine receptor, or an adhesion molecule) present in atherosclerotic lesions, wherein the biological system delivers the therapeutic agent to atherosclerotic lesions in blood vessels. In another embodiment, the biological system comprises: (a) an imaging agent and (b) a carrier that comprises at least one targeting moiety that interacts with a target molecule (e.g., a chemokine ligand or a chemokine receptor) present in atherosclerotic lesions, wherein the biological system delivers the imaging agent to atherosclerotic lesions in blood vessels.

Targeting of the atherosclerotic lesions (stable atherosclerotic plaques or vulnerable atherosclerotic plaques) may be achieved by supplying a biological system that comprises: (a) a single carrier with multiple targeting moieties that interact with two or more target molecules present in atherosclerotic lesions (stable atherosclerotic plaques or vulnerable atherosclerotic plaques), or (b) multiple carriers, each with different targeting moieties that interact with target molecules present in atherosclerotic lesions (stable atherosclerotic plaques or vulnerable atherosclerotic plaques). In both cases, the therapeutic agent or imaging agent in the biological system is effectively delivered to atherosclerotic lesions. Optionally, multiple different targeting moieties bind to each other (e.g., a chemokine and a chemokine receptor) and present an amplified signal reflecting the biological consequences of increased level of the targeting moiety, leading to increased delivery of therapeutic agents or imaging agents to the atherosclerotic lesions.

In a preferred embodiment, the biological systems of the invention deliver a therapeutic agent to vulnerable plaques in blood vessels. The phrase “vulnerable plaque,” as used herein, refers to an atherosclerotic plaque that has a greater chance of rupturing or eroding so as to lead to thrombus formation and therefore to an acute coronary syndrome (ACS) characterized by unstable angina or myocardial infarction, to a stroke, or to a transient cerebral ischemic attack. These types of plaques are also associated with sudden death. In certain cases, a therapeutic agent can prevent plaque erosion or rupture (and thereby prevent thrombus formation and partial or total occlusion of the vessel) by, for example, reducing the lipid contained in the plaque and/or by reducing the inflammatory processes contributing to plaque instability. A therapeutic agent can also prevent further enlargement of an atherosclerotic lesion or prevent occlusion of a blood vessel by an atherosclerotic lesion. In many instances, the therapeutic agents can control or reduce the size of an atherosclerotic lesion.

In one embodiment, the biological systems of the invention deliver an imaging agent to the luminal surface and/or to the interior core of atherosclerotic lesions and render these atherosclerotic lesions visible using appropriate detection methods, such as MRI and cardiac CT. It is a purpose of this invention to identify the specific can be diagnosed by visualizing the atherosclerotic lesion by using the present methods and delivery systems. A therapeutic agent can then be delivered to those vulnerable lesions. Alternatively, conventional treatments may be employed (e.g., stent insertion, angioplasty, or aggressive pharmacologic therapy) after the vulnerable plaques are identified by the present imaging methods.

In all embodiments of the present invention, the targeting moiety in the biological system will be selected with reference to the method (e.g., method of therapy, diagnosis, imaging) in which it will be used. For example, if the severity, extent or severity and extent of vulnerable atherosclerotic lesions/plaques is to be determined, the biological system will include a targeting moiety that interacts with a target molecule present in vulnerable atherosclerotic lesions. If a therapeutic moiety is to be delivered to vulnerable atherosclerotic plaque, the biological system will include a targeting moiety that interacts with a target molecule present in vulnerable atherosclerotic plaque. If the severity, extent or severity and extent of stable atherosclerotic lesions/plaques is to be determined, the biological system will include a targeting moiety that interacts with a target molecule present in stable atherosclerotic lesions/plaques.

### I. Biological Delivery Systems

One aspect of the invention provides biological systems for use in treating, preventing or imaging atherosclerotic lesions (stable atherosclerotic plaques or vulnerable atherosclerotic plaques). Certain embodiments of the invention provide a biological system that comprises: (a) a therapeutic agent or an imaging agent, and (b) a first carrier that comprises at least one targeting moiety that interacts with a target molecule present in atherosclerotic lesions (stable atherosclerotic plaques, vulnerable atherosclerotic plaques). The biological system delivers the therapeutic agent or imaging agent to atherosclerotic lesions (stable atherosclerotic plaques or vulnerable atherosclerotic plaques) in blood vessels. It is understood that the targeting moieties can be used with a wide variety of carriers, including nanoparticles or liposomes containing therapeutic agents as described below.

The term “targeting moiety,” as used herein, refers to a moiety capable of interacting with a target molecule (e.g., a ligand or a receptor) present at the atherosclerotic lesions. Targeting moieties having limited cross-reactivity are preferred. In certain embodiments, suitable targeting moieties include, for example, a member of a specific binding pair, for example, antibodies, proteins, fusion proteins, receptors, ligands, aptamers, homing peptides, and peptidomimetics. A targeting moiety of the subject delivery system results in an increased presence of therapeutic agents or imaging agents at the atherosclerotic lesion sites (stable atherosclerotic plaques or vulnerable atherosclerotic plaques).

Atherosclerotic plaques, and vulnerable plaques in particular, are the site of local inflammatory responses. One of the major mechanisms by which leukocytes are attracted to inflammatory sites is through the expression at the inflammatory site of adhesion molecules and chemotactic molecules called chemokines. The adhesion molecules mediate cell adhesion, and the chemokines mediate cell migration by interacting with chemokine receptors located on the cells; these receptors are characterized by their configuration of cysteine residues with their amino acid sequences (e.g., CCR, CXCR, or CX3CR) expressed on leukocytes. The vulnerable plaque will therefore contain adhesion molecules induced by endothelial cell injury, and both the chemokines induced by inflammation and the chemokine receptors, which are on the cells that have homed to the region of inflammation by interacting with the adhesion molecules and the chemokines.

Accordingly, exemplary targeting moieties include, but are not limited to, ligands such as CXCL1 (growth-regulated oncogene-alpha); CXCL5 (ENA-78); CXCL8 (IL-8); CXCL9 (Mig); CXCL10 (IP-10); CXCL11 (I-TAC); CXCL12 (SDF-1); CCL2 (MCP-1); CCL3 (MIP-1alpha); CCL5 (RANTES); CCL7 (MCP-3); CCL17 (TARC); CCL22 (MDC); fractalkine (FKN); HSP60, (S)-2-amino-4-phosphonobutanoic acid and (S)-3,4-dicarboxyphenylglycine); IL-6, IL-8, IFN-γ, and TNF-α. Target molecules can also include adhesion molecules, such as VCAM-1 and ICAM-1. Other examples of targeting moieties include receptors that bind to any one of these ligands, such as CXCR3, CXCR4, CCR4, CX3CR1, toll like receptors, and metabotropic glutamate receptors (mGluRs). Further examples of targeting moieties include antibodies which bind a ligand or a receptor as described herein. It is understood that fragments or fusion proteins of these ligands or receptors can be produced as targeting moieties provided that they bind to a target molecule present at the atherosclerotic lesions. Osteopontin can also be a target molecule.

In certain specific embodiments, the biological system comprises two or more targeting moieties. These multiple targeting moieties may be present on the same carrier or on different carriers. Preferably, these multiple targeting moieties interact with different target molecules. It is anticipated that greater sensitivity and/or specificity will occur with multiple targeting moieties than with one targeting moiety. For example, two or more antibodies are employed as targeting moieties, and they recognize and bind to multiples target molecules (e.g., such as receptors or ligands) within or near to the atherosclerotic plaque.

As described above, targeting moieties may be antibodies. The term “antibody,” as used herein, includes a monoclonal antibody, a polyclonal antibody, antibody fragments, derivatives or analogs thereof, including without limitation: Fv fragments, single chain Fv (scFv) fragments, Fab′ fragments, F(ab′)2 fragments, single domain antibodies, camelized antibodies and antibody fragments, humanized antibodies and antibody fragments, and multivalent versions of the foregoing; multivalent targeting moieties including without limitation: monospecific or bispecific antibodies, such as disulfide stabilized Fv fragments, scFv tandems ((scFv)₂ fragments), diabodies, tribodies or tetrabodies, which typically are covalently linked or otherwise stabilized (e.g., leucine zipper or helix stabilized) scFv fragments; receptor molecules which naturally interact with a desired target molecule.

Preparation of Antibodies May be Accomplished by Methods Known to Those of skill in the art, such as methods for generating monoclonal antibodies. These methods typically include the step of immunizing of animals, such as mice, with a desired immunogen (e.g., a desired target molecule or fragment thereof). Once the animals have been immunized, and preferably boosted one or more times with the desired immunogen(s), monoclonal antibody-producing hybridomas may be prepared and screened according to well known methods (see, for example, Kuby, Janis, Immunology, Third Edition, pp. 131-139, W.H. Freeman & Co. (1997), for a general overview of monoclonal antibody production, that portion of which is incorporated herein by reference).

Over the past several decades, antibody production has become extremely robust. In vitro methods that combine antibody recognition and phage display techniques allow one to amplify and select antibodies with specific binding capabilities. See, for example, Holt, L. J. et al., “The Use of Recombinant Antibodies in Proteomics,” Current Opinion in Biotechnology, 2000, 11:445-449, incorporated herein by reference. These methods typically are much less cumbersome than preparation of hybridomas by traditional monoclonal antibody preparation methods. Binding epitopes may range in size from small organic compounds such as bromo uridine and phosphotyrosine to oligopeptides on the order of 7-9 amino acids in length.

A targeting moiety need not originate from a biological source. A targeting moiety may, for example, be screened from a combinatorial library of synthetic peptides. One such method is described in U.S. Pat. No. 5,948,635, incorporated herein by reference, which describes the production of phagemid libraries having random amino acid insertions in the pIII gene of M13. These phages may be clonally amplified by affinity selection.

In certain embodiments, it may be desirable to mutate the binding region of a polypeptide targeting moiety (e.g., a chemokine or a chemokine receptor) and select for a targeting moiety with superior binding characteristics as compared to the un-mutated targeting moiety. For example, mutants or fragments of a chemokine receptor may be selected for its superior binding affinity for a chemokine present at atherosclerotic lesions. This may be accomplished by any standard mutagenesis technique, such as by PCR with Taq polymerase under conditions that cause errors. In such a case, the PCR primers could be used to amplify scFv-encoding sequences of phagemid plasmids under conditions that would cause mutations. The PCR product may then be cloned into a phagemid vector and screened for the desired specificity.

In other embodiments, the targeting moieties may be modified to make them more resistant to cleavage by proteases. For example, the stability of a targeting moiety comprising a polypeptide may be increased by substituting one or more of the naturally occurring amino acids in the (L) configuration with D-amino acids. In various embodiments, at least 1%, 5%, 10%, 20%, 50%, 80%, 90% or 100% of the amino acid residues of targeting moiety may be of the D configuration. The substitution of D amino acids for L amino acids neutralizes the digestion capabilities of many of the ubiquitous peptidases found in the digestive tract. Alternatively, enhanced stability of a targeting moiety comprising a peptide bond may be achieved by the introduction of modifications of the traditional peptide linkages. For example, the introduction of a cyclic ring within the polypeptide backbone may confer enhanced stability in order to reduce the effect of many proteolytic enzymes known to digest polypeptides in the stomach or other digestive organs and in serum. In still other embodiments, enhanced stability of a targeting moiety may be achieved by intercalating one or more dextrorotatory amino acids (such as, dextrorotatory phenylalanine or dextrorotatory tryptophan) between the amino acids of targeting moiety. In exemplary embodiments, such modifications increase the protease resistance of a targeting moiety without affecting the activity or specificity of the interaction with a desired target molecule.

In certain embodiments, targeting moieties such as the antibodies, or variants thereof, may be modified to make them less immunogenic when administered to a subject. For example, if the subject is human, the antibody may be “humanized”, such as by transplanting the complimentarily determining region(s) of the hybridoma-derived antibody has been transplanted into a human monoclonal antibody (e.g., as described in Jones, P. et al. (1986), Nature, 321, 522-525 or Tempest et al. (1991), Biotechnology, 9, 266-273). Transgenic mice, or other mammals, may also be used to express humanized antibodies. Such humanization may be partial or complete.

In certain embodiments, a targeting moiety of the invention may be a fusion protein (e.g., a chemokine or a chemokine receptor fused with an Fc fragment). Such fusion protein may contain a tag that facilitates its isolation, immobilization, identification, or detection and/or which increases its solubility. In one embodiment, the fusion protein comprises an Fc fragment of antibodies. The Fc fragment can bind to a Protein A or Protein G. In another preferred embodiment, the fusion protein comprises a homing peptide which selectively directs the fusion protein to a target tissue. The fusion protein may contain other tags, for example, glutathione S-transferase (GST), calmodulin-binding peptide, thioredoxin, maltose binding protein, HA, myc, poly arginine, poly H is, poly His-Asp or FLAG tags. In various embodiments, a targeting moiety of the invention may comprise one or more tags, including multiple copies of the same tag or two or more different tags. It is also within the scope of the invention to include a spacer (such as a polypeptide sequence or a chemical moiety) between a targeting moiety of the invention and the tag in order to facilitate construction or to optimize its structural constraints. In another embodiment, the tagged moiety may be constructed so as to contain protease cleavage sites between the tag and the moiety in order to remove the tag. Examples of suitable endoproteases for removal of a tag, include, for example, Factor Xa and TEV proteases.

In certain embodiments, a targeting moiety of the present invention may comprise a homing peptide which selectively directs a carrier to atherosclerotic lesions (e.g., stable atherosclerotic plaques, vulnerable atherosclerotic plaques). An example of such homing peptide is described in PCT Publication No. WO 03/014145 (incorporated herein by reference). Further, homing peptides for a target tissue (or organ) can be identified using various methods well known in the art. An exemplary method is the in vivo phage display method. Specifically, random peptide sequences are expressed as fusion peptides with the surface proteins of phage, and this library of random peptides are infused into the systemic circulation. After infusion into host mice, target tissues or organs are harvested, the phage is then isolated and expanded, and the injection procedure repeated two more times. Each round of injection includes, by default, a negative selection component, because the injected virus can either randomly bind to tissues or specifically bind to non-target tissues. Virus sequences that specifically bind to non-target tissues will be quickly eliminated by the selection process, while the number of non-specific binding phage diminishes with each round of selection. Many laboratories have identified the homing peptides that are selective for vasculature of brain, kidney, lung, skin, pancreas, intestine, uterus, adrenal gland, retina, muscle, prostate, or tumors. See, for example, Samoylova et al., 1999, Muscle Nerve, 22:460; Pasqualini et al., 1996, Nature, 380:364; Koivunen et al., 1995, Biotechnology, 13:265; Pasqualini et al., 1995, J. Cell Biol., 130:1189; Pasqualini et al., 1996, Mole. Psych., 1:421, 423; Rajotte et al., 1998, J. Clin. Invest., 102:430; Rajotte et al., 1999, J. Biol. Chem., 274:11593. See, also, U.S. Pat. Nos. 5,622,699; 6,068,829; 6,174,687; 6,180,084; 6,232,287; 6,296,832; 6,303,573; 6,306,365.

In yet other embodiments, the targeting moiety may be a peptidomimetic. By employing, for example, scanning mutagenesis to map the amino acid residues of a protein which is involved in binding other proteins, peptidomimetic compounds can be generated which mimic those residues which facilitate the interaction. Such mimetics may then be used as a targeting moiety to deliver a carrier to a target tissue. For instance, non-hydrolyzable peptide analogs of such residues can be generated using benzodiazepine (e.g., see Freidinger et al. in Peptides: Chemistry and Biology, G. R. Marshall ed., ESCOM Publisher: Leiden, Netherlands, 1988), azepine (e.g., see Huffman et al. in Peptides: Chemistry and Biology, G. R. Marshall ed., ESCOM Publisher: Leiden, Netherlands, 1988).

2. Carriers

In certain embodiments, the biological systems of the invention comprise one or more carriers for delivering a therapeutic agent or an imaging agent. The term “carrier,” as used herein, refers to any vehicles, molecules, devices, or molecular complexes that are capable of transporting a therapeutic agent or an imaging agent to the atherosclerotic lesion sites. Examples of the carriers include, but are not limited to, a liposome, a micelles, cells, viral particles, viruses, nanoparticles (e.g., nanospheres) or microparticles (e.g., microspheres), chambered micro-devices, emulsions, lipid discs, polymers, gadolinium-conjugated molecules, superparamagnetic iron oxide particles, multimodal perfluorocarbon nanoparticles, and microbubbles.

One specific example of the carriers is liposomes. Liposomes are small vesicles composed of lipids arranged in spherical bilayers. Liposomes are usually classified as small unilamellar vesicles (SUV), large unilamellar vesicles (LUV), or multi-lamellar vesicles (MLV). SUVs and LUVs, by definition, have only one bilayer, whereas MLVs contain many concentric bilayers (see, e.g., Stryer, Biochemistry, 2d Edition, W.H. Freeman & Co., p. 213 (1981)). Liposomes may be prepared by a variety of techniques (see, e.g., Szoka et al., Ann. Rev. Biophys. Bioeng. 9:467 (1980); U.S. Pat. No. 5,631,018). Liposomes are generally prepared from phospholipids and generally contain cholesterol. In one embodiment, the liposomes comprise non-polymerized or minimally polymerized phospholipids. In another embodiment, the liposomes are prepared by polymerization of double and triple bond-containing monomeric phospholipids. Examples of polymerizable functional groups, include but are not limited to olefins, acetylenes, acrylates and thiols. The liposomes may be polymerized by a variety of techniques known to those skilled in the art including, but not limited to, free radical initiation and ultraviolet and gamma irradiation. Suitable phospholipids are known to those skilled in the art, and include, but are not limited to, phosphatidylcholines, sphingomyelins, phosphatidylglycerols, phosphatidylinositols, phosphatidylserines, phosphatidic acids, DODPC (1,2-di(2,4-Octadecadienoyl)-3-phosphatidylcholine), 2,4-diene phospholipids, di-yne phospholipids. See e.g., U.S. Pat. Nos. 4,485,045 and 4,861,521, which are incorporated herein by reference in their entireties.

Liposomes suitable for use in the composition of the present invention include those composed primarily of vesicle-forming lipids. Vesicle-forming lipids can form spontaneously into bilayer vesicles in water, as exemplified by the phospholipids. The liposomes can also include other lipids incorporated into the lipid bilayers, e.g., cholesterol, with the hydrophobic moiety in contact with the interior, hydrophobic region of the bilayer membrane, and the head group moiety oriented toward the exterior, polar surface of the bilayer membrane.

Another specific example of the carriers includes lipidic micelles, such as micelles composed of PEG-DSPE for use with hydrophobic drugs (see e.g., U.S. Publication No. 20020192275, which is incorporated by reference herein in its entirety).

Another specific example of the carriers is cells. Generally, a nucleic acid encoding a therapeutic agent is introduced into the cells for expression. A targeting moiety can be conjugated on the surface of the cells by methods such as cell painting technology (see e.g., U.S. Pat. No. 6,316,256). Alternatively, the cells are engineered to express a targeting moiety (e.g., a chemokine ligand or receptor). In certain cases, the cells can be progenitor cells that are suitable for delivery to the atherosclerotic lesions, including any totipotent stem cell, pluripotent stem cell, and multipotent stem cell, as well as any of their lineage descendant cells. The progenitor cell may derive from either embryonic tissues or adult tissues. One example of such cells includes marrow-derived stromal cells (MSCs). Methods and vectors for delivering a nucleic acid into a cell are well known in the art (also see below under “Therapeutic Agents”).

3. Therapeutic Agents

In certain embodiments, the invention contemplates delivering any therapeutic agent available to one of skill in the art for treating or preventing atherosclerosis and cardiovascular diseases associated with atherosclerosis. In the present context, the phrase “a therapeutic agent” also comprises active metabolites and prodrugs thereof. An active “metabolite” is an active derivative of a therapeutic agent produced when the therapeutic agent is metabolized. A “prodrug” is a compound that is either metabolized to a therapeutic agent or is metabolized to an active metabolite(s) of a therapeutic agent. This invention can be used to administer therapeutic agents such as small molecular weight compounds, radionuclides, drugs, enzymes, peptides and/or proteins with biological activity, antibodies, nucleic acids or genes that encode therapeutic polypeptides, expression vectors or other nucleic acid constructs, for example, naked plasmid DNAs, any vector carrying one or more genes, any sense or antisense RNA, any ribozyme, or any siRNA for RNA interference (RNAi) purposes.

The therapeutic agents may be linked to a targeting moiety directly or indirectly, and upon administration, these therapeutic agents will become localized at the site of atherosclerotic lesions (e.g., stable atherosclerotic plaques, vulnerable atherosclerotic plaques) and will help control, diminish or otherwise facilitate improved arterial blood flow in the region of the atherosclerotic lesion, as well as help control, diminish or otherwise facilitate removal of cholesterol from the plaque, thereby inhibiting or aiding in inhibiting rupture or erosion of a vulnerable plaque, which would inhibit or aid in inhibition of thrombus formation.

For example, therapeutic agents of the invention include fibrinolytic agents, for example, thrombolytic agents, such as streptokinase, tissue plasminogen activator, plasmin, urokinase, a tissue factor protease inhibitor, a nematode-extracted anticoagulant protein, a metalloproteinase inhibitor, an anti-inflammatory agent(s), a statin, HDL (e.g., the major protein of HDL, apo A1 or a peptide component of apo A1 that has therapeutic activity similar to that of HDL; mutant apo A1, such as apoA1 Milano or other mutant form that has similar therapeutic activity). The therapeutic agent could also be a nucleic acid encoding any of the above agents.

Other specific examples of therapeutic agents include, but are not limited to the following: (a) agents that reduce lipid levels in atherosclerotic lesions, such as an HMG-CoA reductase inhibitor, a thyromimetic, a fibrate, or an agonist of peroxisome proliferator-activated receptors (PPAR); (b) agents that reduce an oxidative process in a mammal such as cytokine-stimulated cyclohydrolase-1 (GTPCH-1) or haptoglobin; (c) agents that modulate expression of an endothelial cell receptor, an endothelial cell adhesion molecule, an endothelial cell integrin, a smooth muscle cell receptor, a smooth muscle cell adhesion molecule or a smooth muscle cell integrin; (d) agents that modulate the proliferation of an endothelial cell or a smooth muscle cell in a mammalian blood vessel; (e) agents that modulate an inflammation associated receptor (or the ligand of such receptor, or the signaling pathway of such a receptor); or inflammation associated transcription factors, such as chemokine receptors, tissue factor (TF); RAGE receptors, toll-like receptors, angiotensin receptors, a TGF receptor, interleukin receptors, a TNF receptor, an IFNγ receptor, a metabotropic glutamate 8 (mGlu8) receptor, receptors that can activate NF-κb; e) agents that modulate an inflammation associated receptor (or the ligand of such receptor, or the signaling pathway of such a receptor); or an inflammation associated transcription factor selected from the group consisting of a chemokine receptor, tissue factor (TF); a RAGE receptor, a toll-like receptor, an angiotensin receptor, a TGF receptor, an interleukin receptor, a TNF receptor, an IFNγ receptor, a metabotropic glutamate 8 (mGlu8) receptor, a receptor that can activate NF-κb; CXCL9 (Mig); CXCL10 (IP-10); CXCL11 (I-TAC); CXCL12 (SDF-1, and its receptor CXCR4); CCL17 (TARC); CCL22 (MDC); CCL3 (MIP-1alpha); CCL5 (RANTES); CCL7 (MCP-3); CXCL1 (growth-regulated oncogene-alpha); CXCL5 (ENA-78); CXCL8 (IL-8); fractalkine (FKN; and its receptor, CX3CR1); HSP60; toll-like receptors; metabotropic glutamate receptors (mGluRs; and its ligands such as (S)-2-amino-4-phosphonobutanoic acid and (S)-3,4-dicarboxyphenylglycine); c-Fos, IL-6, IL-8, MCP-1, IFN-γ, and TNF-α; and adhesion molecules such as VCAM-1 and ICAM-1; (f) agents that modulate proliferation, apoptosis or necrosis of endothelial cells, vascular smooth muscle cells, lymphocytes, monocytes, or neutrophils; (g) agents that modulate production, degradation, or crosslinking of an extracellular matrix protein such as collagen, a elastin, or a proteoglycan; (h) agents that modulate activation, secretion or lipid loading of a cell within a mammalian blood vessel; (i) agents that modulate activation or proliferation of a dendritic cell or monocyte/macrophage cell within a mammalian blood vessel; (j) agents that modulate activation or adhesion of a platelet at a mammalian blood vessel wall or could otherwise inhibit clot formation, such as activated protein C (APC); and (k) an agent consisting of a nucleic acid that encodes a protein therapeutic agent, wherein the protein therapeutic agent has an in vivo activity that is beneficial to a mammal suffering from an atherosclerotic lesion.

In one embodiment, the therapeutic agent is a nucleic acid that encodes a protein whose activity can benefit a mammal (such as a human) suffering from an atherosclerotic lesion. The gene therapy agent can, for example, reduce the size of a lesion, prevent platelet interaction with the lesion, reduce or prevent the growth of smooth muscle cells, reduce the likelihood of a vulnerable plaque eroding or rupturing or otherwise stabilize or beneficially interact with the atherosclerotic lesion. In another embodiment, the therapeutic agent is a nucleic acid that can generate an antisense RNA useful for reducing the expression of a deleterious protein at the site of the atherosclerotic lesion. Such therapeutic nucleic acids can be directly attached to a targeting moiety of the invention, or it can be present in a phage particle, liposome or other transformation vector available to one of skill in the art.

In another embodiment, a nucleic acid encoding a polypeptide therapeutic agent is directly administered in vivo, where it is targeted to the site of atherosclerotic lesions via linkage to a targeting moiety. Optionally, a “naked” DNA is linked to a targeting moiety and can be directly delivered, for example, by use of controlled pressure-mediated delivery methods (see, e.g., von der Leyen, Braun-Dullaeus, et al, Hum. Gene Ther. 1999, 10:2355). Such methods provide safe and efficient arterial transfer to cells at the site of atherosclerotic lesions (e.g., stable atherosclerotic plaques, vulnerable atherosclerotic plaques) for nucleic acids, genes and oligonucleotides.

In another embodiment, a nucleic acid encoding a targeting moiety (e.g., a chemokine ligand or receptor) is joined with another nucleic acid that encodes a biologically active therapeutic agent to form a hybrid or recombinant nucleic acid. The hybrid or recombinant nucleic acid is incorporated into an appropriate vector that is expressed in a cell type of interest. Cells containing the resulting vector are then delivered to the atherosclerotic lesions (e.g., stable atherosclerotic plaques, vulnerable atherosclerotic plaques) by in vivo or ex vivo administrations.

As an example, therapeutic agents include genes or nucleic acids that encode proteins or antisense RNAs that inhibit inflammatory events at the sites of atherosclerosis lesion progression or at sites of vulnerable atherosclerotic lesions. Such genes or nucleic acids include the dominant-negative form or soluble forms of the chemokine receptors (any of CCR, CXCR, or CX3CR) and adhesion molecules such as VCAM-1 and ICAM-1. Likewise, dominant-negative forms or soluble forms of toll-like receptors (e.g., TLR-1, TLR-2, TLR-3, TLR-4 or TLR-5) may be therapeutic agents of the invention. As another example, therapeutic agents include genes or nucleic acids that encode proteins or antisense RNAs that inhibit foam cell formation and thus retard progression and/or stimulate regression of atherosclerotic lesions. Such genes or nucleic acids can, for example, encode secreted “decoys” or mutants of macrophage scavenger receptors MSR (sMSR).

In certain embodiments, a therapeutic agent is a nucleic acid encoding an antisense RNA. Such an antisense RNA is typically a “sense” DNA sequence cloned into an expression cassette in the opposite orientation relative to its normal orientation. When operably linked to a promoter in the expression cassette in such an opposite orientation, an RNA that is complementary to the natural mRNA encoded by the nucleic acid is synthesized.

In certain embodiments, a therapeutic agent is a double-stranded RNA (dsRNA) that can trigger silencing of homologous gene expression by a mechanism termed RNAi (for RNA-mediated interference) (Fire et al., 1998, Nature 391, 806-811). RNAi is an evolutionarily conserved phenomenon and a multistep process that involves generation of active small interfering RNA (siRNA) in vivo through the action of an RNase III endonuclease, Dicer. The resulting 21- to 23-nt siRNA mediates degradation of the complementary homologous RNA (Bernstein et al., 2001, RNA 7, 1509-1521; Sharp et al., 2001, Genes Dev. 15, 485-490). Such RNAi technology could be employed with the current invention to block expression of a specific gene by employing the RNAi. RNAs of interest whose expression can be blocked include those that encode the same target proteins, genes, RNA and DNA as described herein.

In certain aspects, targeted delivery of a therapeutic agent to atherosclerotic plaque(s) can enhance endothelial coverage and healing at the site of atherosclerosis, especially in plaques that are vulnerable to rupturing and to producing thrombosis, unstable angina, myocardial infarction or stroke. Other areas that could benefit from such targeted delivery to heal endothelium include the sites of vascular interventions such as angioplasty or stenting.

In certain embodiments, nucleic acids encoding a therapeutic agent or a targeting moiety of the invention can be included in expression cassettes and/or expression vectors. To prepare expression cassettes for transformation, the nucleic acid encoding a therapeutic agent may be circular or linear, double-stranded or single-stranded. Any vectors available to one of skill in the art can be employed for this purpose, including viral (adenovirus, retrovirus, lentivirus or other viruses) vectors or synthetic vectors (such as liposomes, microparticles or nanoparticles) to allow improved delivery of genes, ribozymes, antisense oligonucleotides, dsRNAs, or DNA to atherosclerotic lesions. A number of vector systems are known for the introduction of foreign or native genes into mammalian cells. These include SV40 virus (Okayama et al., 1985); bovine papilloma virus (DiMaio et al., 1982); adenovirus (Morin et al., 1987; Dai et al., 1995; Yang et al., 1996; Tripathy et al., 1996; Quantin et al., 1992; Rosenfeld et al., 1991; Wagner, 1992; Curiel et al., 1992; Curiel, 1991; LeGal LaSalle et al., 1993; Kass-Eisler et al., 1993); adeno-associated virus (Muzyczka, 1994; Xiao et al., 1996); herpes simplex virus (Geller et al., 1988; Huard et al., 1995; U.S. Pat. No. 5,501,979); lentivirus (Douglas, et al., Hum Gene Ther. 12(4):401-413 (2001), Miyoshi, et al., Virol. 72:8150-8157 (1999), Garvey et al. Virology, 175:391-409, 1990, Berkowitz et al. J. Virol. 7(7):33713382 (2001); WO 01/44458, U.S. Pat. Nos. 6,277,633 and 5,380,830.

Any such vector may contain an inducible promoter operably linked to a coding region for a polypeptide therapeutic agent. Such a promoter allows controllable expression of the nucleic acid through an appropriate inducer of transcription. A vector can be in the form of chimeric DNA that contains the coding region of the selected nucleic acid flanked by control sequences that promote the expression of the nucleic acid within target cells. As used herein, “chimeric” means that a vector comprises DNA from at least two different species, or comprises DNA from the same species, which is linked or associated in a manner that does not occur in the “native” or wild type of the species. The term “control sequences” is defined to mean DNA sequences necessary for the expression of an operably linked coding region in a particular host organism. Control sequences that are suitable for eukaryotic cells include promoters, polyadenylation signals, and enhancers. Further, other elements may be included in the nucleic acid or vector as desired by one of skill in the art to obtain the optimal performance of the transforming nucleic acid or vector in the cell. The phrase “operably linked” is defined to mean that the nucleic acids are placed in a functional relationship with another nucleic acid sequence. For example, control sequences are operably linked to a nucleic acid encoding a beneficial protein; a promoter or enhancer is operably linked to a coding region if it affects the transcription of the coding region; or a ribosome binding site is operably linked to a coding region if it is positioned so as to facilitate translation.

In other embodiments, the present invention relates to delivering pharmaceutical compounds (drugs) as therapeutic agents to sites of atherosclerotic lesions (e.g., stable atherosclerotic plaques, vulnerable atherosclerotic plaques) or to sites of revascularization procedures including stenting or access ports for dialysis. Such pharmaceutical compounds can be directly linked to one or more targeting moieties. Alternatively, they can be incorporated into a chemical composition containing one or more targeting moieties. In another embodiment, they can be incorporated into an artificial carrier (e.g. a liposome or other microparticle) that contains one or more targeting moieties. Examples of such drug therapies include compounds that regulate HMG-CoA reductase (e.g., statins), fibrates, and other compounds affecting PPARs (e.g., PPAR alpha, gamma, and/or delta agonists), and thyromimetics.

In certain specific embodiments, the therapeutic agents of the present invention include HDL, a peptide component of HDL that has similar therapeutic actions as HDL (e.g., the binding of cholesterol and its transportation to tissues involved in its degradation, or binding of a receptor involved in cholesterol transport, such as ABCA1 and SR-B1), and a nucleic acid encoding a peptide/protein component of HDL. Increased HDL levels have been associated with a protective effect on atherogenesis, and increasing HDL in hypercholesterolemic animals has been shown to reduce atherosclerosis. Accordingly, the present invention relates to delivering nucleic acid (DNA, RNA) encoding a native peptide/protein component of HDL, apoA-I Milano, or an apoA-I mimetic peptide to the atherosclerotic lesions (e.g., vulnerable plaques) such that the HDL reduces the size of the existing lesions and stabilizes plaques at risk of rupture. The transgene(s) encoding a native peptide/protein of HDL, apoA-I Milano, or an apoA-I mimetic peptide can be introduced via any carriers as described above (e.g., a cell and a liposome). See e.g., Brewer et al., (2004)Arterioscler. Thromb. Vasc. Biol., 24:1755-1760; Brewer (2004) Arterioscler. Thromb. Vasc. Biol. 24:387-381; and Brewer (2004) N. Engl. J. Med. 350:1491-1494.

In one specific embodiment, the invention provides a biological system for delivering an HDL therapeutic an individual. The biological system comprises: (a) an HDL therapeutic selected from HDL, apoA-1, a mutant apoA-1, an apoA-1 mimetic peptide, and a nucleic acid encoding either apoA-1 or a peptide mimetic of apoA-1; and (b) a carrier from which the HDL therapeutic of (a) is delivered to blood vessels. For example, the apoA-1 protein or an apoA-1-mimetic peptide binds to a receptor involved in cholesterol transport, such as the ATP-binding cassette transporter 1 (ABCA1) or the SR-B1 receptor. Optionally, the carrier is selected from a nanoparticle, a microparticle, a cell, and a liposome. Optionally, the nucleic acid HDL therapeutic is DNA in an expression vector.

In another specific embodiment, the invention provides a delivery system that targets atherosclerotic plaque in blood vessels. The delivery the system comprises: (a) an HDL therapeutic selected from HDL, apoA-1, a mutant apoA-1, an apoA-1 mimetic peptide, and a nucleic acid encoding either apoA-1 or a peptide mimetic of apoA-1; and (b) a carrier that interacts/binds with a constituent of atherosclerotic plaque and delivers the HDL therapeutic of (a) to the atheroslerotic plaque. For example, the apoA-1 protein or an apoA-1-mimetic peptide binds to a receptor involved in cholesterol transport, such as the ATP-binding cassette transporter 1 (ABCA1) or the SR-B1 receptor. Optionally, the carrier is selected from a nanoparticle, a microparticle, a cell, and a liposome. Optionally, the nucleic acid HDL therapeutic is DNA in an expression vector. To illustrate, the carrier bears on its surface a binding partner of the constituent of atherosclerotic plaque, such as a constituent of the plaque itself (including molecules residing on or expressed by cells normally residing in the plaque, cells that have migrated to the plaque, extracellular matrix of the plaque, blood vessels residing in the plaque, such as vasa vasorum), or a product released/shed from the plaque. In certain cases, the atherosclerotic plaque is vulnerable to rupture.

In yet another specific embodiment, the invention provides an expression vector comprising: (a) DNA that encodes a protein component of HDL selected from apoA-1, a mutant apoA-1, an apoA-1 mimetic peptide; and (b) a promoter specifically expressed in blood vessels that contain atherosclerotic plaques, wherein the DNA of (a) is operably linked to the promoter of (b). Optionally, the expression vector comprises a promoter specifically expressed in blood vessels that contain atherosclerotic plaques that are vulnerable. Merely to illustrate, one example of such a promoter would be a promoter that contains binding sites for activators usually associated with inflammatory situations (such as the promoter for MCP-1); another example would be a promoter that contains binding sites for activators usually associated with newly growing blood vessels (such as the promoter for VEGF).

4. Imaging Agents

In certain embodiments, the invention contemplates delivering any imaging agent available to one of skill in the art for detecting and diagnosing atherosclerotic lesions. Examples of the imaging agents include, but are not limited to, a radioactive agent; a contrast agent; a magnetic agent or a paramagnetic agent; a liposome, a micelle, a cell, a viral particle, a virus, a microsphere (e.g., a microsphere), a nanosphere (e.g., a nanosphere), a chambered micro-device, an emulsion, a lipid disc, a polymer, gadolinium-conjugated molecules, superparamagnetic iron oxide particles, multimodal perfluorocarbon nanoparticles, and microbubbles.

For example, the radioactive agent is radioiodine, technetium, yttrium, or other radiopharmaceutical. For example, the contrast agent is gadolinium, manganese, barium sulfate, an iodinated or noniodinated agent, an ionic agent or nonionic agent, superparamagnetic iron oxide particles, or multimodal perfluorocarbon nanoparticles. For example, the liposome carries a radioactive agent, a contrast agent, or any other imaging agent, including, but not limited to gadolinium, manganese, barium sulfate, an iodinated or noniodinated agent, an ionic agent or nonionic agent, superparamagnetic iron oxide particles, or multimodal perfluorocarbon nanoparticles. For example, the detecting agent encoded by the expression vector or virus is a fluorescent polypeptide (e.g., luciferase).

It is understood that the invention contemplates any imaging agent that has been employed for CT, fluoroscopy, SPECT imaging, optical imaging, PET, MRI or gamma imaging.

In certain embodiments, an imaging agent is directly linked to a targeting moiety and delivered to the site of atherosclerotic lesions. Optionally, an imaging agent is incorporated in a carrier (e.g., a liposome, a nanoparticle, etc.) which comprises a targeting moiety.

### II. Therapeutic Uses

Certain aspects of the invention relate to methods of treating atherosclerosis or a cardiovascular disease associated with atherosclerosis in an individual. If it is administered prior to clinical manifestation of the unwanted condition (e.g., cardiovascular disease, atherosclerosis, heart attack or stroke), the treatment is prophylactic, e.g., it reduces (totally or partially) the extent to which the condition develops. If administered after manifestation of the unwanted condition, the treatment is therapeutic (i.e., it is intended to diminish, ameliorate or maintain the existing unwanted condition or side effects therefrom). In preferred embodiments, the individual is a human.

One specific aspect provides a method of slowing the development of atherosclerosis in an individual. Another specific aspect provides a method of treating or preventing atherosclerosis in an individual. Such methods comprise administering to an individual in need thereof a therapeutically effective amount of a biological system comprising (a) a therapeutic agent and (b) a carrier that comprises a targeting moiety that interacts with a target molecule present in atherosclerotic lesions, wherein the biological system delivers the therapeutic agent to atherosclerotic lesions in blood vessels. The phrase “therapeutically effective amount” means that amount of such a substance that produces a desired local or systemic effect at a reasonable benefit/risk ratio applicable to any treatment. In certain embodiments, a therapeutically effective amount of a compound will depend on its therapeutic index, solubility, and the like. For example, certain compounds discovered by the methods of the present invention may be administered in a sufficient amount to produce a reasonable benefit/risk ratio applicable to such treatment.

Generally, the atherosclerotic lesions are associated with a condition such as plaque rupture, plaque erosion, acute coronary syndrome, stroke, transient ischemia attack, heart attack, angina, unstable angina, thrombosis, myocardial infarction, ischemic heart disease, coronary artery disease, peripheral artery disease, or transplantation-induced sclerosis. Thus, the term “cardiovascular disease associated with atherosclerosis” includes references to any of the above conditions or diseases that are medically linked to atherosclerosis in that they are a consequence of atherosclerotic lesions. “Coronary artery disease” (“CAD”) is a pathological state characterized by atherosclerotic involvement of the coronary arteries. This initially is mild and the minimal narrowing of the artery it produces does not impair coronary flow and thus does not lead to myocardial ischemia and is asymptomatic. Nonetheless, such plaques can suddenly erode or rupture and lead to ACS. As the lesions progress, they produce sufficient arterial obstruction such that ischemia occurs, at which time the disease may become symptomatic (e.g., angina pectoris and myocardial infarction). As used herein, CAD includes both symptomatic or asymptomatic disease. The same considerations relate to atherosclerotic involvement of the arterial vessels that supply the brain and the legs. Plaque erosion or rupture occurs most commonly in lesions that are relatively mild and do not yet impair blood flow. As a result, severe manifestation of atherosclerosis (heart attack, stroke, sudden death) can occur as presenting manifestations of the disease, without prior, less severe symptoms having developed.

In some embodiments of the methods described herein, the individual is afflicted with cardiovascular atherosclerosis, cerebrovascular atherosclerosis, peripheral vessel atherosclerosis, coronary heart atherosclerosis or a combination thereof. In other embodiments, the subject is afflicted with obesity, insulin resistance, diabetes, hypertension, hypercholesterolemia, or a combination thereof.

In one specific embodiment, the invention provides a method of slowing the development of atherosclerotic lesions in an individual, comprising administering to the individual a therapeutically effective amount of a biological system which comprises: (a) an HDL therapeutic selected from HDL, apoA-1, a mutant apoA-1, an apoA-1 mimetic peptide, and a nucleic acid encoding either apoA-1 or a peptide mimetic of apoA-1; and (b) a carrier from which the HDL therapeutic of (a) is delivered to blood vessels.

In another specific embodiment, the invention provides a method of reducing cholesterol levels in atherosclerotic plaque in an individual in need thereof, comprising administering to the individual a therapeutically effective amount of a delivery system which comprises: (a) an HDL therapeutic selected from HDL, apoA-1, a mutant apoA-1, an apoA-1 mimetic peptide, and a nucleic acid encoding either apoA-1 or a peptide mimetic of apoA-1; and (b) a carrier that interacts/binds with a constituent of atherosclerotic plaque and delivers the HDL therapeutic of (a) to the atheroslerotic plaque.

In certain embodiments, the subject methods of the invention further comprise administering to the patient a therapeutically effective amount of at least one additional agent, such as a statin (such as atorvastatin, cerivastatin, fluvastatin, lovastatin, mevastatin, pravastatin, rosuvastatin, sinivastatin), fibrate (such as beclobrate, bezafibrate, ciprofibrate, clinofibrate, clofibrate), clofibride, etofylline clofibrate, fenofibrate, genifibrozil, pirifibrate, plafibribe, sinifibrate, tocofibrate, a bile-acid binding resin, colesevelain, colestipol, colestyramine, divistyramine, a nicotinate, acipimox, binifibrate, etofibrate, niceritrol, nicofibrate, pirozadil, ronifibrate, sorbinicate, tocoferil nicotinate, an omega triglyceride, an omega acid ethyl ester, an omega marine triglycerides, an acyl-coenzymeA cholesterol O-acyl transferase (ACAT) inhibitor, avasimibe, a PPAR gamma agonist (e.g., pioglitazone), a cholesterol absorption inhibitor (e.g., ezetimibe), a lipase inhibitor, and/or listat.

In some embodiments of the methods described herein, the biological system comprising one or more therapeutic agents can be formulated as pharmaceutical compositions and administered to a mammalian host (such as a human) in a variety of dosage forms adapted to the chosen route of administration. For example, the biological system can be administered to an individual (or subject) intravenously, intramuscularly, intradermally, subcutaneously, by means of a stent or a combination thereof. In other embodiments, the biological system comprising one or more therapeutic agents is administered systemically, or administered locally at the site of an atherosclerotic plaque.

Solutions of the biological system can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

In certain embodiments, the subject therapeutic methods may be combined with another type of therapeutic agent for cardiovascular diseases, including, but not limited to, streptokinase, tissue plasminogen activator, plasmin, urokinase, a tissue factor protease inhibitor, a nematode-extracted anticoagulant protein, a metalloproteinase inhibitor, and an anti-inflammatory agent.

### III. Diagnosis and Imaging Uses

In certain aspects, the invention relates to diagnostic and prognostic methods for atherosclerosis and methods of predicting the likelihood of development of symptomatic atherosclerosis or a cardiovascular disease associated with atherosclerosis in an individual, preferably a human. The term “diagnostic” refers to assays that provide results which can be used by one skilled in the art, typically in combination with results from other assays, to determine if an individual is suffering from a disease or disorder of interest such as atherosclerosis. The term “prognostic” refers to the use of assays to evaluate the likelihood an individual having such a disease or disorder will develop a complication of the disease, such as sudden death, heart attack or stroke). The term “pharmacogenetic” refers to the use of assays to predict which individual patients in a group will best respond to a particular therapeutic or prophylactic composition or treatment.

In one specific embodiment, the invention provides a method of delivering an imaging agent to atherosclerosis lesions in an individual, comprising administering to the individual an effective amount of a biological system comprising (a) an imaging agent and (b) a carrier that comprises a targeting moiety that interacts with a target molecule present in atherosclerotic lesions, wherein the biological system delivers the imaging agent to atherosclerotic lesions in blood vessels. The biological system can be delivered to the individual by a variety of routes, such as by intravenous administration.

In another specific embodiment, the invention provides a method of assessing the severity of an atherosclerotic lesion in an individual, comprising: 1) administering a biological system comprising (a) an imaging agent and (b) a carrier that comprises a targeting moiety that interacts with a target molecule present in atherosclerotic lesions; and 2) observing the amount, localization, shape, density, or relative distribution of the imaging agent on an atherosclerotic lesion in the individual.

It is understood that the subject methods provided herein can be used to diagnose the location, extent, and pathologic composition of atherosclerotic lesions anywhere within the body of an individual. For example, detection of an imaging agent which is delivered to the atherosclerotic lesions can provide information regarding the location, shape, extent and pattern of the lesion. Optionally, the subject methods can detect atherosclerotic lesions of different stages and thus can be used to diagnose the staging or severity of the lesions and potential risk of thrombosis. In certain cases, the imaging agent is delivered to, into and/or across a luminal surface of vascular endothelium.

In certain embodiments, the present invention relates to performing physical imaging of an individual. “Physical imaging,” as used herein, refers to imaging of all or a part of an individual's body. Physical imaging can be positive, that is, can be used to detect the presence of a specific type of atherosclerotic lesions (e.g., vulnerable plaques). In another embodiment, positive physical imaging can be used to detect the presence or absence of stable atherosclerotic lesions/plaques. Alternatively, the physical imaging can be “negative.” That is, it can be used to detect the absence of a specific type of atherosclerotic lesion (e.g., vulnerable plaques). Both positive and negative physical imaging permits visualization and/or detection of both normal and of abnormal pathology such as atherosclerotic lesions. Optionally, such imaging methods can be used to quantify or determine the extent, size, and/or number of an atherosclerotic lesion. Thus, an estimate can be made of the extent of disease, facilitating, for example, clinical diagnosis and/or prognosis.

For physical imaging, an imaging agent is administered to the individual. The imaging agent is linked to one or more targeting moieties either directly or indirectly, wherein the targeting moieties bind to and localize to atherosclerotic lesions. In certain cases, the biological system comprising an imaging agent is administered to the individual (e.g., intravenously or intra-arterially), and then the individual is assessed for the presence or absence or concentration of the imaging agent in blood vessels. A “concentration,” as used herein, is an amount of the imaging agent at a particular location in the individual's body. If the concentration is greater than would be expected from mere circulation or diffusion of the imaging agent in the individual, the individual is determined to have atherosclerotic lesions. A concentration is indicative of delivery of the agent to the target sites (e.g., atherosclerotic lesions).

In certain embodiments, the imaging methods of the present invention further comprise delivering a therapeutic agent with the imaging agent to the atherosclerotic lesions.

### INCORPORATION BY REFERENCE

All publications and patents mentioned herein are hereby incorporated by reference in their entirety as if each individual publication or patent was specifically and individually indicated to be incorporated by reference.

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

We claim:

1. A biological system for delivering a therapeutic agent to atherosclerotic lesions in blood vessels in an individual, the system comprising: (a) a therapeutic agent and (b) a carrier that comprises at least one targeting moiety that interacts with a target molecule present in atherosclerotic lesions, wherein the biological system delivers the therapeutic agent to atherosclerotic lesions in blood vessels.

2. The biological system of claim 1, wherein the biological system comprises two or more targeting moieties.

3. The biological system of claim 2, wherein the two or more targeting moieties are different.

4. The biological system of claim 3, wherein the two or more targeting moieties interact with different target molecules.

5. The biological system of claim 1, wherein the target molecule is a ligand selected from the group consisting of: CXCL1 (growth-regulated oncogene-alpha); CXCL5 (ENA-78); CXCL8 (IL-8); CXCL9 (Mig); CXCL10 (IP-10); CXCL11 (I-TAC); CXCL12 (SDF-1); CCL2 (MCP-1); CCL3 (MIP-1alpha); CCL5 (RANTES); CCL7 (MCP-3); CCL17 (TARC); CCL22 (MDC); fractalkine (FKN); HSP60, (S)-2-amino-4-phosphonobutanoic acid and (S)-3,4-dicarboxyphenylglycine); IL-6, IL-8, IFN-γ, TNF-α, VCAM-1, ICAM-1 and osteopontin.

6. The biological system of claim 5, wherein the targeting moiety is a receptor of the ligand.

7. The biological system of claim 5, wherein the targeting moiety is an antibody to the ligand.

8. The biological system of claim 1, wherein the target molecule is a receptor of a ligand selected from the group consisting of: CXCL1 (growth-regulated oncogene-alpha); CXCL5 (ENA-78); CXCL8 (IL-8); CXCL9 (Mig); CXCL10 (IP-10); CXCL11 (I-TAC); CXCL12 (SDF-1); CCL2 (MCP-1); CCL3 (MIP-1alpha); CCL5 (RANTES); CCL7 (MCP-3); CCL17 (TARC); CCL22 (MDC); fractalkine (FKN); HSP60, (S)-2-amino-4-phosphonobutanoic acid and (S)-3,4-dicarboxyphenylglycine); IL-6, IL-8, IFN-γ, TNF-α; VCAM-1, ICAM-1 and osteopontin.

9. The biological system of claim 8, wherein the targeting moiety is a ligand of the receptor.

10. The biological system of claim 8, wherein the targeting moiety is an antibody to the receptor.

11. The biological system of claim 8, wherein the receptor is selected from the group consisting of: CXCR3, CXCR4, CCR4, CX3CR1, toll-like receptors, metabotropic glutamate receptors (mGluRs), and the receptors to VCAM-1 and ICAM-1.

12. The biological system of claim 1, further comprising a therapeutic agent and at least one binding partner that interacts with a component/constituent of the atherosclerotic lesions.

13. The biological system of claim 1, wherein the therapeutic agent is selected from the group consisting of: a nucleic acid, a polypeptide, an antibody, a small molecule compound, and a peptidomimetic.

14. The biological system of claim 13, wherein the nucleic acid consists of naked DNA or is inserted as a transgene into a viral vector.

15. The biological system of claim 1, wherein the carrier is selected from the group consisting of a liposome, a micelle, a cell, a viral particle, a virus, a microparticle, a nanoparticle, a chambered micro-device, an emulsion, a lipid disc, a polymer, gadolinium-conjugated molecules, superparamagnetic iron oxide particles, multimodal perfluorocarbon nanoparticles, and microbubbles.

16. The delivery system of claim 1, wherein the atherosclerotic lesions are vulnerable atherosclerotic plaques.

17. A biological system for delivering an imaging agent to atherosclerotic lesions in blood vessels in an individual, the system comprising (a) an imaging agent and (b) a carrier that comprises at least one targeting moiety that interacts with a target molecule present in atherosclerotic lesions, wherein the biological system delivers the imaging agent to atherosclerotic lesions in blood vessels.

18. The biological system of claim 17, wherein the biological system comprises two or more targeting moieties.

19. The biological system of claim 18, wherein the two or more targeting moieties are different.

20. The biological system of claim 19, wherein the two or more targeting moieties interact with different target molecules.

21. The biological system of claim 17, wherein the imaging agent is selected from the group consisting of a radioactive agent; a contrast agent; a magnetic agent or a paramagnetic agent; a liposome, a micelle, a cell, a viral particle, a virus, a microparticle, a nanoparticle, a chambered micro-device, an emulsion, a lipid disc, a polymer, gadolinium-conjugated molecules, superparamagnetic iron oxide particles, multimodal perfluorocarbon nanoparticles, and microbubbles.

22. The biological system of claim 21, wherein the radioactive agent is radioiodine, technetium, yttrium, or other radiopharmaceutical.

23. The biological system of claim 21, wherein the contrast agent is gadolinium, manganese, barium sulfate, an iodinated or noniodinated agent, an ionic agent or nonionic agent, superparamagnetic iron oxide particles, or multimodal perfluorocarbon nanoparticles.

24. The biological system of claim 17, wherein the carrier is selected from the group consisting of a liposome, a micelle, a cell, a viral particle, a virus, a microparticle, a nanoparticle, a chambered micro-device, an emulsion, a lipid disc, a polymer, gadolinium-conjugated molecules, superparamagnetic iron oxide particles, multimodal perfluorocarbon nanoparticles, and microbubbles.

25. The biological system of claim 17, further comprising at least one therapeutic agent.

26. A method of slowing the development of atherosclerosis in an individual, comprising administering to the individual a therapeutically effective amount of a biological system of claim 1.

27. The method of claim 26, wherein the individual is a human.

28. A method of treating or preventing atherosclerosis in an individual, comprising administering a therapeutically effective amount of a biological system of claim 1.

29. The method of claim 28, wherein the atherosclerosis causes plaque rupture, plaque erosion, acute coronary syndrome, stroke, transient ischemia attack, heart attack, angina, unstable angina, thrombosis, myocardial infarction, ischemic heart disease, peripheral artery disease, or transplantation-induced sclerosis.

30. The method of claim 28, further comprising administering a second therapeutic agent selected from the group consisting of streptokinase, tissue plasminogen activator, plasmin, urokinase, a tissue factor protease inhibitor, a nematode-extracted anticoagulant protein, a metalloproteinase inhibitor, and an anti-inflammatory agent.

31. The method of claim 28, wherein the individual is a human.

32. A method of delivering an imaging agent to atherosclerosis lesions in an individual, comprising administering to the individual an effective amount of a biological system of claim 1.

33. A method of identifying the severity of an atherosclerotic lesion in an individual, comprising: 1) administering a biological system comprising (a) an imaging agent and (b) a carrier that comprises a targeting moiety that interacts with a target molecule present in atherosclerotic lesions; and 2) observing the amount, localization, shape, density, or relative distribution of the imaging agent on an atherosclerotic lesion in the individual.

34. A biological system for delivering an HDL therapeutic an individual, wherein the biological system comprises: (a) an HDL therapeutic selected from HDL, apoA-1, a mutant apoA-1, an apoA-1 mimetic peptide, and a nucleic acid encoding either apoA-1 or a peptide mimetic of apoA-1; and (b) a carrier from which the HDL therapeutic of (a) is delivered to blood vessels.

35. A delivery system that targets atherosclerotic plaque in blood vessels, the system comprising (a) an HDL therapeutic selected from HDL, apoA-1, a mutant apoA-1, an apoA-1 mimetic peptide, and a nucleic acid encoding either apoA-1 or a peptide mimetic of apoA-1; (b) a carrier that interacts/binds with a constituent of atherosclerotic plaque and delivers the HDL therapeutic of (a) to the atheroslerotic plaque.

36. The delivery system of claim 35, wherein the carrier bears on its surface a binding partner of the constituent of atherosclerotic plaque.

37. A method of slowing the development of atherosclerotic lesions in an individual, comprising administering to the individual a therapeutically effective amount of a biological system of claim 34, whereby the development of atherosclerotic lesions is slowed in the individual.

38. A method of reducing cholesterol levels in atherosclerotic plaque in an individual in need thereof, comprising administering to the individual a therapeutically effective amount of a delivery system of claim 35, whereby cholesterol levels in atherosclerotic plaque are reduced in the individual.

39. An expression vector comprising: (a) DNA that encodes a protein component of HDL selected from apoA-1, a mutant apoA-1, an apoA-1 mimetic peptide; and (b) a promoter specifically expressed in blood vessels that contain atherosclerotic plaques, wherein the DNA of (a) is operably linked to the promoter of (b).
